(19) United States
(12) Patent Application Publication
(10) Pub. No.: US 2026/0272288 A1
(43) Pub. Date: Sep. 17, 2026

(54) TRANSOCULAR DIAGNOSTIC DEVICE AND METHOD FOR PARKINSON’S DISEASE CAUSED BY SUBSTANTIA NIGRA DOPAMINERGIC NEURODEGENERATION

(71) Applicant: ZHEJIANG UNIVERSITY, Zhejiang (CN)

(72) Inventors: Peng LI, Zhejiang (CN); Kaiyuan LIU, Zhejiang (CN)

(73) Assignee: ZHEJIANG UNIVERSITY, Zhejiang (CN)

(21) Appl. No.: 19/676,780

(22) Filed: May 13, 2026

Related U.S. Application Data
(63) Continuation of application No. PCT/CN2024/116488, filed on Sep. 3, 2024.

(30) Foreign Application Priority Data
Jun. 12, 2024 (CN) 202410752522.0

(51) Int. Cl. A61B 3/12; A61B 3/00; A61B 5/00; G06T 7/00; G06T 7/62

(52) U.S. Cl. CPC A61B 3/1233; A61B 3/0008; A61B 3/0025; A61B 3/1241; A61B 5/4082; A61B 5/4848; G06T 7/0012; G06T 7/62; G06T 2207/10101; G06T 2207/30101

(57) ABSTRACT

The invention discloses a transocular diagnostic device and method for Parkinson's disease caused by substantia nigra dopaminergic neurodegeneration. An optical vision device stimulates the eyes of a subject before and after a specific condition of increased dopamine levels in the body, and at the same time records the stimulation index. A fundus blood flow detection device detects and records fundus blood vessel and blood flow parameter change information before and after the increase in dopamine levels in the body of the subject and before and after visual stimulation to generate a blood flow index. A signal processing device receives the blood flow index and the stimulation index for processing to obtain a retinal neurovascular coupling function index; a computer or a chip receives the retinal neurovascular coupling function index to determine a risk or an outcome of substantia nigra dopaminergic neurodegeneration of a brain.

### CROSS-REFERENCE TO RELATED APPLICATION

This application is a continuation of international application of PCT application serial no. PCT/CN2024/116488, filed on Sep. 3, 2024, which claims the priority benefit of China application no. 202410752522.0, filed on Jun. 12, 2024. The entirety of each of the above-mentioned patent applications is hereby incorporated by reference herein and made a part of this specification.

### BACKGROUND OF THE INVENTION

### Field of the Invention

The invention relates to the field of brain information detection techniques, specifically to a transocular diagnostic device and method for Parkinson's disease (PD) caused by substantia nigra dopaminergic neurodegeneration.

### Description of Related Art

Damage to dopaminergic neurons in the substantia nigra region of the brain may lead to motor symptoms and severely impact quality of life. Therefore, there is an urgent need for a reliable system working method that may enable early detection of substantia nigra dopaminergic neurodegeneration. Existing positron emission tomography (PET) and single-photon emission computed tomography (SPECT) based on brain neuroimaging may specifically detect substantia nigra dopaminergic neurodegeneration of the brain, but the use of isotope tracers poses a potential radiation hazard, and the longer detection time and higher detection cost limit the further application thereof. The retina is an extension of the brain, and tests on the retina may reflect substantia nigra dopaminergic neurodegeneration of the brain. Since degeneration of dopaminergic neurons in the substantia nigra leads to a decrease in retinal thickness and vascular density, optical coherence tomography (OCT) and optical coherence tomography angiography (OCTA) may currently be used to detect the above. However, these methods for detecting dopaminergic neurodegeneration based on retinal structural biomarkers have very limited sensitivity and specificity.

Therefore, it is of great significance to utilize the unique window of the retina to detect dopaminergic neuron damage in the substantia nigra region of the brain with high sensitivity and specificity.

### SUMMARY OF THE INVENTION

The objective of this invention is to address the shortcomings of existing techniques by proposing a transocular diagnostic device and method for PD caused by substantia nigra dopaminergic neurodegeneration.

This invention applies stimulation to the subject to activate retinal neural activity, uses the eye as a detection window to collect and record data, and thus may quickly, non-invasively, with high sensitivity and high specificity, detect substantia nigra dopaminergic neurodegeneration of the brain.

The objective of the invention is achieved through the following technical solution:

I. A transocular diagnostic device for Parkinson's disease caused by substantia nigra dopaminergic neurodegeneration:
The system includes an optical vision device for performing an optical visual stimulation on an eye of a subject before and after a specific condition of increased dopamine levels in a body of the subject, and at the same time recording a stimulation index S during the optical visual stimulation and sending the stimulation index S to a signal processing device;
The system further comprises a fundus blood flow detection device, which is a retinal blood vessel and blood flow detection device used to detect and record fundus blood vessel and blood flow parameter change information before and after the specific condition of increased dopamine levels in the body of the subject, and before and after a visual stimulation, to generate a blood flow index B and send the blood flow index B to the signal processing device;
The system further comprises the signal processing device receiving the blood flow index B from the fundus blood flow detection device and the stimulation index S from the optical vision device for analyzing and processing to obtain the retinal neurovascular coupling function index R containing dopamine-related information;
The system further comprises a computer or a chip receiving the retinal neurovascular coupling function index R from the signal processing device, and analyzing the retinal neurovascular coupling function index R to determine a risk or an outcome of substantia nigra dopaminergic neurodegeneration of a brain.

To obtain a general risk of substantia nigra dopaminergic neurodegeneration, only stimulation is needed without specific conditions to enable a general functional assessment of substantia nigra dopaminergic neurodegeneration. However, to obtain accurate results of substantia nigra dopaminergic neurodegeneration, it is necessary to combine the application of the specific condition and the application of the stimulus in order to achieve accurate functional testing of substantia nigra dopaminergic neurodegeneration.

The specific condition includes temporarily increasing the dopamine levels in the body, and the increased dopamine levels in the body may be achieved by taking dopamine-related drugs, and the dopamine-related drugs are drugs that regulate the dopamine content in the body of the subject.

The optical vision device emits specific light into the eye of the subject through a visually perceptible means such as a display or a lamp to stimulate the retinal neural activity of the subject. By applying visual light stimulation, retinal neural activity is activated to induce a hyperemia response, and the retinal neurovascular coupling function index may be obtained by detection. For example, the optical vision device may use LEDs to illuminate the eye of the subject once every few seconds to hundreds of seconds, but is not specifically limited thereto.

More specifically, the optical visual stimulation mode includes flashing light stimulation or pattern stimulation;

Furthermore, the color of the flashing light includes monochromatic visible light or polychromatic visible light, and further includes green light or white light; the frequency, the duty cycle, and the illuminance of the flashing light are adjustable;

Furthermore, the pattern includes a checkerboard pattern or a sine wave pattern; the contrast and the reversal frequency of the checkerboard pattern are adjustable; the contrast and the spatial frequency of the sine wave pattern are adjustable;

The light-emitting device includes an LED, a laser, or a computer screen.

This invention is based on the changes in blood vessels and blood flow signals when retinal neural activity changes (retinal neurovascular coupling function index) to reflect substantia nigra dopaminergic neurodegeneration of the brain.

The optical visual stimulation provided by the optical vision device before and after the subject experiences the specific condition of increased dopamine levels may be the same or different. The preferred scenarios are the same.

The fundus blood flow detection device records and collects fundus blood vessel and blood flow parameter change information in real time at at least one moment from each of a pre-stimulation stage L0 to a post-stimulation stage L1 before and after a specific condition of increased dopamine levels in the body of the subject, thereby generating the blood flow index B. Typically, the record is a multiple sampling record of the entire process of the pre-stimulation stage L0 and the post-stimulation stage L1, or information data from several intervals at multiple points of the interval time period in the entire discrete sampling process.

More specifically, the fundus blood flow detection device includes an optical coherence tomography (OCT) system, or a dynamic vascular analyzer, or a laser Doppler flowmeter, or a fluorescence angiography system, or an ultrasound detector;

The fundus blood flow detection device has a synchronization device for automatically synchronizing stimulation triggering with fundus signal recording; or the fundus blood flow detection device does not have a synchronization device and the stimulation triggering and the fundus signal recording are manually controlled.

The pre-stimulation stage L0 refers to the period before the stimulus begins.

The post-stimulation stage L1 mainly consists of a stimulation segment containing only the complete stimulation after the stimulation initiation time, or consists of a stimulation segment containing only the complete stimulation after the stimulation initiation time and a recovery segment without stimulation after the stimulation.

The drug is typically a drug increasing the dopamine levels in the body, and may further include a dopamine precursor (such as levodopa), or a dopamine compound (such as dopamine hydrochloride), or a tyrosine hydroxylase inhibitor (such as carbidopa), or a dopamine reuptake inhibitor (such as theophylline), or a combination of several of the above drugs.

The signal processing device has an embedded software module. After receiving the blood flow index B and the stimulation index S, the software module processes the data to obtain the retinal neurovascular coupling function index R, and the retinal neurovascular coupling function index R is then sent to a computer/chip. According to the retinal neurovascular coupling function index R, the risk of brain lesions or the result of substantia nigra dopaminergic neurodegeneration is determined.

The computer or the chip determines a risk level of substantia nigra dopaminergic neurodegeneration of the brain according to amplitude attenuation features/conditions or time delay features/conditions extracted from the retinal neurovascular coupling function index and correlated with dopamine levels;

The computer or the chip determines an accurate result of substantia nigra dopaminergic neurodegeneration of the brain according to whether the amplitude attenuation features/conditions or the time delay features/conditions extracted from the retinal neurovascular coupling function index after increased dopamine levels in the body are reversible, i.e., reversibility.

Fundus vascular and blood flow parameters include a vascular size, a blood flow velocity, and a blood flow volume;

Further including a vessel diameter and a vessel density in an OCTA image;

Further including a mean of blood flow region decorrelation coefficients and a sum of the blood flow region decorrelation coefficients in an OCTA signal.

The fundus blood vessel and blood flow parameter change information refers to information of the above parameters.

The stimulation index S specifically refers to a dose of the optical visual stimulation;

The blood flow index B includes absolute changes in the fundus vessels and blood flow parameters before and after the optical visual stimulation, and/or relative changes in the fundus vessels and blood flow parameters before and after the optical visual stimulation; the changes usually referred to are a ratio or a difference between the parameter values of the post-stimulation stage L1 and the parameter values of the pre-stimulation stage L0. And/or includes a peak amplitude, a peak time (peak time refers to the moment corresponding to the peak amplitude), and a combination of the peak amplitude and the peak time of the fundus blood vessel and blood flow parameter change information before and after the optical visual stimulation.

The retinal neurovascular coupling function index R is a combination of the blood flow index B and the stimulation index S, expressed as: R=B/S. In particular, a maximum value of the retinal neurovascular coupling function index R is the amplitude, and a time corresponding to the amplitude is the delay time.

To determine the risk level of substantia nigra dopaminergic neurodegeneration of the brain, the control system operates as follows:
In a natural state DO before increased dopamine levels in the body of the subject, the optical visual stimulation is applied to the subject using the optical vision device, a retinal neurovascular coupling function index R0 under the natural state DO is calculated by the signal processing device according to the blood flow index B obtained by the fundus blood flow detection device respectively before and after the optical visual stimulation, and the stimulation index S of the optical stimulation device, and results are then determined by the computer or the chip;
If a peak amplitude A0 in the retinal neurovascular coupling function index R0 is less than or equal to a preset first peak amplitude threshold TA1, i.e., amplitude attenuation, amplitude attenuation features/conditions occurred, and it is determined as high risk of substantia nigra dopaminergic neurodegeneration; otherwise, it is determined as low risk;
Alternatively, if a peak time TO in the retinal neurovascular coupling function index R0 is greater than or equal to a preset first peak time threshold TT1, i.e., time delay, time delay features/conditions occurred, and it is determined as high risk of substantia nigra dopaminergic neurodegeneration; otherwise, it is determined as low risk;
Alternatively, if the peak amplitude A0 in the retinal neurovascular coupling function index R0 is less than or equal to the preset first peak amplitude threshold TA1, and the peak time T0 is greater than or equal to the preset first peak time threshold TT1, amplitude attenuation features/conditions and time delay features/conditions occurred simultaneously, and then it is determined as high risk of substantia nigra dopaminergic neurodegeneration; otherwise, it is determined as low risk.

To determine whether there is substantia nigra dopaminergic neurodegeneration of the brain, the control system continues to operate as follows when determining a high risk of substantia nigra dopaminergic neurodegeneration:
Further induced under the specific condition of increased dopamine levels in the body of the subject, and then in a state D1 of increased dopamine of the subject, the subject is subjected to another optical visual stimulation by the optical vision device, then the retinal neurovascular coupling function index R1 in the state D1 of increased dopamine is calculated according to the blood flow index B obtained by the fundus blood flow detection device respectively before and after the optical visual stimulation, and the stimulation index S of the optical stimulation device by the signal processing device, and then the determination is made by the computer or the chip; If a peak amplitude A1 in the retinal neurovascular coupling function index R1 under the state D1 of increased dopamine is greater than or equal to the preset second peak amplitude threshold TA2, i.e., amplitude attenuation is reversed, amplitude attenuation features/conditions are reversed, and it is determined that there is substantia nigra dopaminergic neurodegeneration; otherwise, it is determined to be absent;
Alternatively, if a peak time T1 in the retinal neurovascular coupling function index R1 under the state D1 of increased dopamine is less than or equal to the preset second peak time threshold TT2, i.e., time delay is reversed, time delay features/conditions are reversed, and it is determined that there is substantia nigra dopaminergic neurodegeneration; otherwise, it is determined to be absent;
Alternatively, if the peak amplitude A1 in the retinal neurovascular coupling function index R1 under the state D1 of increased dopamine is greater than or equal to the second peak amplitude threshold TA2, and the peak time T1 thereof is less than or equal to the second peak time threshold TT2, both the amplitude attenuation features/conditions and the time delay features/conditions are reversed simultaneously, and it is determined that there is substantia nigra dopaminergic neurodegeneration; otherwise, it is determined to be absent.

If the risk of substantia nigra dopaminergic neurodegeneration is determined to be low, the step of applying specific conditions to the subject is not needed.

The peak amplitude threshold and the peak time threshold under the state of increased dopamine in the body may be the same as or different from the threshold before the state of increased dopamine.

Each of the above thresholds may be obtained by establishing a classification model using clinical data.

II. A transocular diagnostic method for Parkinson's disease caused by substantia nigra dopaminergic neurodegeneration:
an optical visual stimulation method of performing an optical visual stimulation on the eye of a subject before and after a specific condition of increased dopamine levels in a body of the subject, and at the same time recording a stimulation index S during the optical visual stimulation;
a fundus blood flow detection method of specifically performing retinal vessel and blood flow detection to generate a blood flow index B before and after the specific condition of increased dopamine levels in the body of the subject, and before and after a visual stimulation;
a quantitative method for a retinal hyperemic response function index R of obtaining the retinal hyperemic response function index R containing dopamine-related information in combination of the blood flow index B and the stimulation index S, comprising an amplitude and a delay time;
a method for assessing a degree of substantia nigra dopaminergic neurodegeneration of assessing the degree of substantia nigra dopaminergic neurodegeneration according to the dopamine-related information contained in the retinal hyperemic response function index R to determine a risk or an outcome of substantia nigra dopaminergic neurodegeneration of a brain.

The optical visual stimulation method is performed through a visually perceptible means such as a display or a lamp to stimulate a retinal neural activity of the subject.

Fundus vascular and blood flow parameters include a vascular size, a blood flow velocity, and a blood flow volume;

Further including a vessel diameter and a vessel density in an OCTA image;

Further including a mean of blood flow region decorrelation coefficients and a sum of the blood flow region decorrelation coefficients in an OCTA signal.

The stimulation index S specifically refers to a dose of the optical visual stimulation;

The blood flow index B includes absolute changes in the fundus vessels and blood flow parameters before and after the optical visual stimulation, and/or relative changes in the fundus vessels and blood flow parameters before and after the optical visual stimulation; the changes usually referred to are a ratio or a difference between the parameter values of the post-stimulation stage L1 and the parameter values of the pre-stimulation stage L0. And/or includes a peak amplitude, a peak time (peak time refers to the moment corresponding to the peak amplitude), and a combination of the peak amplitude and the peak time of the fundus blood vessel and blood flow parameter change information before and after the optical visual stimulation.

The retinal neurovascular coupling function index R is a combination of the blood flow index B and the stimulation index S, expressed as: R=B/S. Here, a maximum value of the retinal neurovascular coupling function index R is the amplitude, and a time corresponding to the amplitude is the delay time.

The specific method for assessing the degree of substantia nigra dopaminergic neurodegeneration is as follows:

If a peak amplitude A0 in the retinal neurovascular coupling function index R0 before increased dopamine levels in the body of the subject is less than or equal to a preset first peak amplitude threshold TA1, or a peak time TO in the retinal neurovascular coupling function index R0 is greater than or equal to a preset first peak time threshold TT1, or the peak amplitude A0 in the retinal neurovascular coupling function index R0 is less than or equal to the preset first peak amplitude threshold TA1 and the peak time TO is greater than or equal to the preset first peak time threshold TT1, amplitude attenuation features/conditions or time delay features/conditions occurred, and it is determined as high risk of substantia nigra dopaminergic neurodegeneration, otherwise, it is determined as low risk; when it is determined as high risk of substantia nigra dopaminergic neurodegeneration, the retinal neurovascular coupling function index R1 under a state D1 of increased dopamine of the subject is further determined;

If a peak amplitude A1 in the retinal neurovascular coupling function index R1 in the state D1 of increased dopamine of the subject is greater than or equal to a preset second peak amplitude threshold TA2, or a peak time T1 in the retinal neurovascular coupling function index R1 in the state D1 of increased dopamine is less than or equal to a preset second peak time threshold TT2, or if the peak amplitude A1 in the retinal neurovascular coupling function index R1 in the state D1 of increased dopamine is greater than or equal to the second peak amplitude threshold TA2 and the peak time T1 thereof is less than or equal to the second peak time threshold TT2, amplitude attenuation features/conditions or time delay features/conditions are reversed, and it is determined that there is substantia nigra dopaminergic neurodegeneration; otherwise, it is determined to be absent.

The beneficial effects of the invention are as follows:

Existing PET and SPECT detection systems based on brain neuroimaging use X-rays as detection signals, which poses a potential radiation hazard. The invention uses light signals or sound signals to detect the retinal neurovascular coupling function index caused by substantia nigra dopaminergic neurodegeneration. It is radiation-free, has better safety, and is suitable for large-scale screening and longitudinal monitoring.

Existing detection systems based on traditional OCT use changes in retinal thickness caused by substantia nigra dopaminergic neurodegeneration as a detection marker. However, the axial resolution of OCT systems is about 4 micrometers to 7 micrometers, and the retinal thinning is only 1 micrometer to 2 micrometers, resulting in poor detection sensitivity. In addition, factors such as aging may also lead to a decrease in retinal thickness, resulting in worse detection specificity.

Existing detection systems based on traditional OCTA use changes in retinal microvessel morphology caused by substantia nigra dopaminergic neurodegeneration as a detection marker. However, factors such as intraocular pressure may also cause changes in retinal microvessels, resulting in worse detection specificity.

### BRIEF DESCRIPTION OF THE DRAWINGS

FIG. 1 is a schematic diagram of a device of the invention.

FIG. 2 is a flowchart of an embodiment of a system working method of the invention.

FIG. 3 is a schematic diagram of an embodiment of a device of the invention.

FIG. 4 shows the detection results of retinal blood flow volume response in healthy and substantia nigra dopaminergic neurodegenerative mice under light stimulation in an untreated state, in an exemplary embodiment of the invention.

FIG. 5 shows the changes in relative retinal blood flow volume induced by light stimulation in healthy and substantia nigra dopaminergic neurodegenerative mice under three states: before administration of levodopa, after administration of levodopa, and after levodopa metabolism, in an exemplary embodiment of the invention.

FIG. 6 shows the classification results of healthy and substantia nigra dopaminergic neurodegenerative mice based on the peak amplitude of retinal blood flow volume induced by light stimulation before administration of levodopa and after administration of levodopa, in an exemplary embodiment of the invention.

### DESCRIPTION OF THE EMBODIMENTS

The specific embodiments of the invention will now be described in detail with reference to the accompanying drawings, and the accompanying drawings form part of this document. It should be noted that these descriptions and examples are merely illustrative and should not be construed as limiting the scope of the invention. The scope of the invention is defined by the appended claims, and any modifications based on the claims of the invention are within the scope of the invention.

To facilitate understanding of the embodiments of the invention, each operation is described as a plurality of discrete operations; however, the order of description does not represent the order in which the operations are performed.

In this description, the sample measurement space is represented by three-dimensional xyz coordinates based on spatial orientation. This description is merely intended to facilitate discussion and is not intended to limit the application of embodiments of the invention. In particular: the depth z-direction is the direction along the incident optical axis; the x-y plane is a plane perpendicular to the optical axis, wherein x and y are orthogonal, and x represents the OCT lateral fast scan direction, and y represents the slow scan direction.

The use of i, t, etc., to represent variables is merely for the objective of facilitating discussion and is not intended to limit the application of the embodiments of the invention. These values may be any numbers such as 1, 2, 3, etc.

The specific implementation process of the system working method of the invention includes the following steps:

Step 1: Before and after the subject experiences an increase in dopamine levels, optical visual stimulation is applied to the eyes of the subject. The stimulation index S during the optical visual stimulation is recorded and sent to the signal processing device.

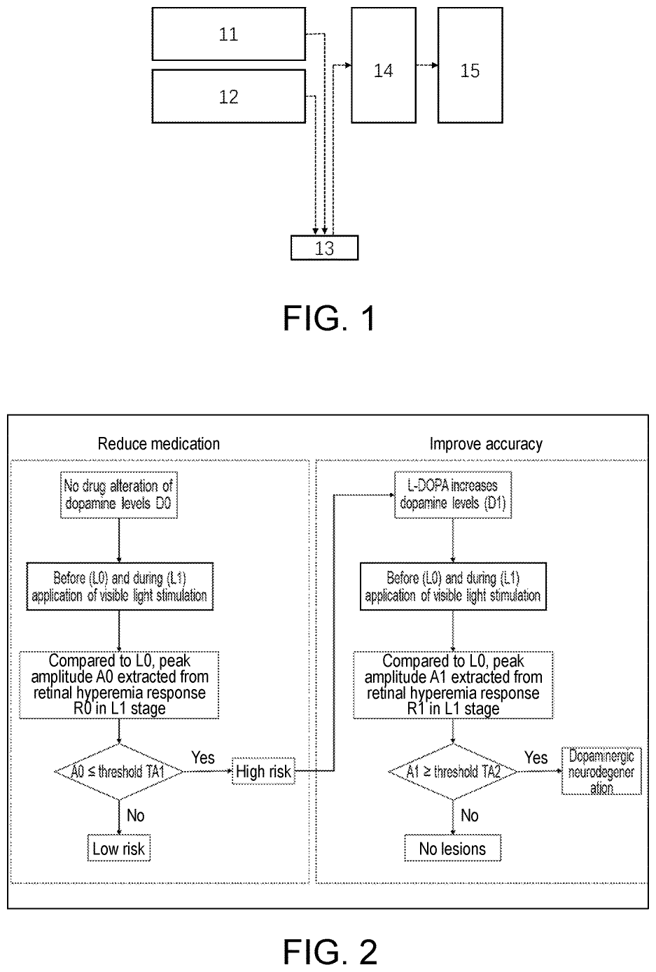

The specific implementation device system is shown in FIG. 1, which mainly consists of two parts: 11-12 and 14-15. The light stimulation module 12 with LED light is used as an optical vision device, the fundus blood flow detection device includes a detection signal generation module 11 and a detection signal acquisition module 14, the eye of the subject is used as a sample 13, and the signal processing module 15 is used as a signal processing device for data processing.

The signal generation module 11 transmits the detection signal to the sample 13, and the light stimulation module 12 generates stimulation light to activate the neuronal activity on the sample 13. Subsequently, the signal detection module 14 collects the signal returned from the sample 13 and transmits the signal to the signal processing module 15 for further processing.

The light stimulation module 12 generates flashing visible light to stimulate the retina of the eye of the sample 13 at a wavelength range of 380 nm to 760 nm, a stimulation time of 30 s, an illuminance of 1000 lux, a frequency of 10 Hz, a duty cycle of 50%, and the stimulation dose thereof corresponds to the stimulation index S. Before and after a specific condition of increased dopamine levels of the subject, the eye of the subject is subjected to the same dose of optical visual stimulation. Step 2: Using drugs to increase dopamine levels in the body of the subject, changes in fundus vascular and blood flow parameters from the pre-stimulation stage L0 to the post-stimulation stage L1 are recorded at two different states: before drug administration DO and after drug administration D1, to obtain the retinal neurovascular coupling function index at the current state. Specifically:

As shown in FIG. 2, the general implementation process of the system of the invention is as follows:

Before drug administration (DO) in which the drug is not taken, stimulation is performed by the optical vision device, and the retinal neurovascular coupling function index is obtained by recording fundus blood vessel and blood flow parameter change information from the pre-stimulation stage L0 to the post-stimulation stage L1 using the fundus blood flow detection device;

The subject then takes the drug, so that the dopamine content in the body of the subject is increased. After drug administration D1, the same stimulation is performed again by the optical vision device. The fundus blood vessel and blood flow parameter change information from the pre-stimulation stage L0 to the post-stimulation stage L1 is recorded by the fundus blood flow detection device to obtain the retinal neurovascular coupling function index.

In practice, the fundus blood flow detection device records the retinal neurovascular coupling function index using clock signal control to synchronize the two processes of stimulation application and retinal neurovascular coupling function index recording, thereby achieving precise control of the recording time node.

The signal processing device may be embedded with a software module. After the fundus blood vessel and blood flow parameter change information from the blood flow detection device is received, the software module processes the data to obtain the retinal neurovascular coupling function index. Then, according to the retinal neurovascular coupling function index, the risk of brain lesions or the result of substantia nigra dopaminergic neurodegeneration may be determined.

As shown in FIG. 2, to determine the risk level of substantia nigra dopaminergic neurodegeneration of the brain, the control system operates as follows:

In the natural state DO before the rise in dopamine levels in the body of the subject, optical visual stimulation is applied to the subject using the optical vision device. The retinal neurovascular coupling function index R0 under the natural state DO without applying any stimulation to the subject is calculated by the signal processing device, according to the blood flow index B obtained by the fundus blood flow detection device respectively before and after optical visual stimulation and the stimulation index S of the optical stimulation device. The result is then determined by the software module within the computer or the chip:
If the peak amplitude A0 in the retinal neurovascular coupling function index R0 is less than or equal to the preset first peak amplitude threshold TA1, i.e., amplitude attenuation, amplitude attenuation features/conditions occurred, and it is determined as high risk of substantia nigra dopaminergic neurodegeneration; otherwise, it is determined as low risk;
Alternatively, if the peak time TO in the retinal neurovascular coupling function index R0 is greater than or equal to the preset first peak time threshold TT1, i.e., time delay, time delay features/conditions occurred, and it is determined as high risk of substantia nigra dopaminergic neurodegeneration; otherwise, it is determined as low risk;
Alternatively, if the peak amplitude A0 in the retinal neurovascular coupling function index R0 is less than the preset first peak amplitude threshold TA1, and the peak time TO is greater than or equal to the preset first peak time threshold TT1, both amplitude attenuation features/conditions and time delay features/conditions occurred simultaneously, and it is determined as high risk of substantia nigra dopaminergic neurodegeneration; otherwise, it is determined as low risk.

As may be seen above, without stimulation, it is impossible to accurately determine whether there is substantia nigra dopaminergic neurodegeneration; only the risk of substantia nigra dopaminergic neurodegeneration may be assessed.

As shown in FIG. 2, to determine whether there is substantia nigra dopaminergic neurodegeneration of the brain, the control system continues to operate as follows when it is determined as high risk of substantia nigra dopaminergic neurodegeneration:

Further, under specific conditions that induce an increase in dopamine levels in the body of the subject, and then in state D1 after the dopamine increase, the subject is subjected to another optical visual stimulation by the optical vision device. The retinal neurovascular coupling function index R1 in state D1 after the dopamine increase is then calculated by the signal processing device, according to the blood flow index B obtained by the fundus blood flow detection device respectively before and after the optical visual stimulation, and the stimulation index S of the optical stimulation device. Lastly, the determination is made by the computer or the chip:

If the peak amplitude A1 in the retinal neurovascular coupling function index R1 under state D1 after dopamine rise is greater than or equal to the preset second peak amplitude threshold TA2, that is, the amplitude attenuation is reversed, the amplitude attenuation features/conditions are reversed, and it is determined that there is substantia nigra dopaminergic neurodegeneration; otherwise, it is determined to be absent;

Alternatively, if the peak time T1 in the retinal neurovascular coupling function index R1 under state D1 after dopamine rise is less than or equal to the preset second peak time threshold TT2, i.e., the time delay is reversed, the time delay features/conditions are reversed, and it is determined that there is substantia nigra dopaminergic neurodegeneration; otherwise, it is determined to be absent;

Alternatively, if the peak amplitude A1 in the retinal neurovascular coupling function index R1 under state D1 after dopamine rise is greater than or equal to the second peak amplitude threshold TA2, and the peak time T1 thereof is less than or equal to the second peak time threshold TT2, both the amplitude attenuation features/conditions and the time delay features/conditions are reversed simultaneously, and it is determined that there is substantia nigra dopaminergic neurodegeneration; otherwise, it is determined to be absent.

In a specific embodiment, fundus blood vessel and blood flow parameter change information is recorded before drug administration (DO) and after drug administration (D1) using a weak coherent interference-based OCT device. The two processes of stimulation application and fundus parameter recording are precisely synchronized using a clock signal. In particular, the post-light stimulation stage L1 includes a light stimulation segment and a non-stimulated recovery segment. The entire process of the pre-light stimulation stage L0 and the stimulation stage L1 is canned using OCT to obtain retinal interference signals at different time points.

After Fourier transform is performed on the interference signal, the retinal structure signal is obtained. The signal is calculated and analyzed using the first-order and zero-order autocovariances to obtain two features: inverse signal-to-noise ratio (I) and decorrelation coefficient (D). Then, an I-D two-dimensional feature space is constructed, and the dynamic blood flow signal is analyzed and determined using a linear classifier. The OCTA blood flow angiography image sequence of the retina in the two states of before drug administration DO and after drug administration D1, in the pre-light stimulation stage L0 and the post-light stimulation stage L1, is obtained. The generated OCTA blood flow angiography image sequence is first binarized to remove noise interference. Then, the binarized OCTA matrix is skeletonized, the intersections of the retinal microvascular skeleton are located and removed, and each blood vessel is obtained as a single connected domain vascular skeleton segment numbered as i. The blood flow volume Flowᵢ(tⱼ) of each blood vessel at each time node is extracted.

The blood flow volume of each blood vessel segment at each time point before light stimulation t0 is averaged to obtain the baseline blood flow volume Flow_baseᵢ of the blood vessel i before the hyperemic response:

$${Flow\_base}_{i} = \frac{1}{N} {\sum }_{i = 1}^{N} {Flow}_{i} ( {t}_{j} )$$

Here, N represents the number of data points collected within the baseline segment t0, t is the time dimension, and Flowᵢ(tⱼ) represents the blood flow volume of the blood vessel i at the time point tⱼ.

The relative change in blood flow volume during the stimulation stage and the recovery stage of the scintillation light is then calculated as the blood flow index B:

$${B}_{i} ( {t}_{k} ) = \frac{{Flow}_{i} ( {t}_{k} ) - {Flow\_base}_{i}}{{Flow\_base}_{i}}$$

Here, the blood flow index Bᵢ(tₖ) is the relative change in blood flow volume in the blood vessel i at the time point tₖ.

In combination of the blood flow index Bᵢ(tₖ) and the stimulation index S, the retinal neurovascular coupling function index Rᵢ(tₖ) of the blood vessel i at the time point t; is obtained:

$${R}_{i} ( {t}_{k} ) = \frac{{B}_{i} ( {t}_{k} )}{S}$$

Here, specifically, the stimulation index S is the stimulation dose under the conditions of 380 nm to 760 nm wavelength, 30 s stimulation time, 1000 lux illuminance, 10 Hz frequency, and 50% duty cycle, but not limited thereto.

The retinal neurovascular coupling function index of all blood vessels in the scanning field of view at the same sampling time point during the entire sampling process is averaged and used as the overall neurovascular coupling function index R of the retina. The maximum value of this functional index in the stimulation stage and the recovery stage is taken as the peak amplitude A, and the peak amplitude A is used as an indicator to determine substantia nigra dopaminergic neurodegeneration of the brain. In the natural state DO before the rise in dopamine levels in the body of the subject, if the peak amplitude A0 in the retinal neurovascular coupling function index R0 is less than or equal to the preset first peak amplitude threshold TA1, it is determined as high risk of substantia nigra dopaminergic neurodegeneration; otherwise, it is determined as low risk.

When it is determined as high risk of substantia nigra dopaminergic neurodegeneration, the subject is given oral levodopa (250 mg) to increase dopamine levels. In state D1 after the dopamine levels of the subject rise, the following determination is made: if the peak amplitude A1 in the retinal neurovascular coupling function index R1 in state D1 after the dopamine rises is greater than or equal to the preset second peak amplitude threshold TA2, then it is determined that there is substantia nigra dopaminergic neurodegeneration; otherwise, it is determined to be absent.

Depending on the different methods of signal detection, devices such as the fundus blood flow detection device shown in FIG. 1 may specifically include:
1) Optical coherence tomography device. The retinal signal is detected by low-coherence interference light, and the signal is received using a spectrometer or a balanced detector to obtain a depth-resolved OCT sample volume.
2) Dynamic vascular analyzer. The retinal signal is directly recorded using a fundus camera, and the signal is received using a CMOS or CCD sensor, thereby obtaining a planar image of the retinal surface.
3) Laser Doppler flowmeter. The retinal signal is detected using a monochromatic infrared laser, and the returned light signal is received by a photodetector, thereby obtaining retinal blood flow information.
4) Fundus fluorescein angiography instrument. A retinal fluorescence sample is obtained by exciting the fundus fluorescence using a specific wavelength and then detecting the emitted fluorescence using a camera.
5) Ultrasonic imaging device. Ultrasonic signals are sent to the retina and ultrasonic signals returning from the retina are received using an ultrasonic transducer, thereby obtaining a depth-resolved ultrasonic sample.

All of the above measuring devices may generate retinal flow imaging by analyzing the signal differences between blood flow and surrounding tissues.

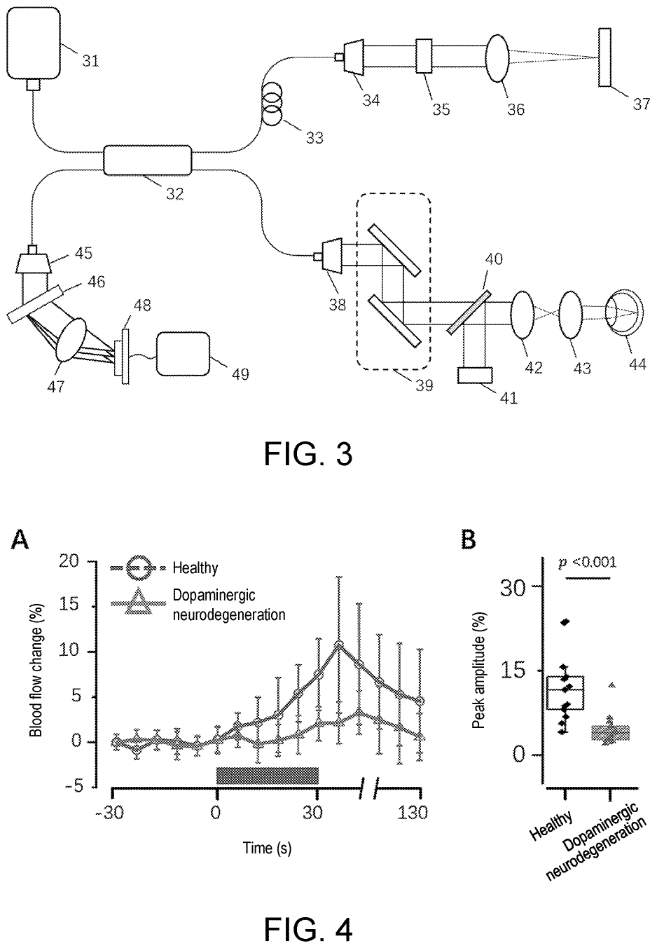

FIG. 3 illustrates one exemplary embodiment of the invention disclosed herein. The system includes a broadband light source 31, an 80:20 fiber optic coupler 32, a polarization controller 33, a reference arm collimator 34, a dispersion matching module 35, a reference arm focusing lens 36, a reference arm reflector 37, a sample arm collimator 38, a sample arm OCT scanning device 39, a sample arm dichroic mirror 40, a visible light stimulation light source 41, a sample arm double cemented lens 42, an eyepiece 43, a sample eye 44, a detection module collimator 45, a grating 46, a focusing lens 47, a high-speed linear array camera 48, and a signal processing module 49.

The broadband light source 31 is connected to one branch of one end of the 80:20 fiber optic coupler 32. Another branch of one end of the 80:20 fiber optic coupler 32 is connected to the signal processing module 49 after passing through the detection module collimator 45, the grating 46, the focusing lens 47, and the linear array camera 48. One branch of the other end of the 80:20 fiber optic coupler 32 is connected to the reference arm. The reference arm includes a polarization controller 33, a reference arm collimating lens 34, a dispersion matching module 35, a reference arm focusing lens 36, and a reference arm reflector 37 arranged in sequence. One branch of the other end of the 80:20 fiber optic coupler 32 is connected to one end of the polarization controller 33, and the other end of the polarization controller 33 is connected to the incident end of the reference arm collimating lens 34. A dispersion matching module 35 and a reference arm focusing lens 36 are arranged sequentially between the reference arm collimating lens 34 and the reference arm reflecting mirror 37. The dispersion matching module 35 is close to the reference arm collimating lens 34, and the reference arm focusing lens 36 is close to the reference arm reflecting mirror 37. One branch of the other end of the 80:20 fiber optic coupler 32 is connected to the sample arm, and the sample arm includes a sample arm collimating lens 38, a sample arm OCT scanning device 39, a sample arm dichroic mirror 40, a sample arm double cemented lens 42, an eyepiece 43, and a sample eye 44 arranged in sequence. Between the sample arm collimating lens 38 and the sample eye 44, the sample arm OCT scanning device 39, the sample arm dichroic mirror 40, the sample arm double cemented lens 42, and the eyepiece 43 are sequentially arranged. The focal points of the eyepiece 43 and the sample arm double cemented lens 42 are aligned, so that parallel light is irradiated onto the ocular surface of the sample eye 44 and focused onto the retina through the eyeball. The stimulation light emitted by the visible light stimulation source 41 is coupled into the sample arm detection light path through the sample arm dichroic mirror 40, and then transmitted to the sample eye 44 after passing through the sample arm double cemented lens 42 and the eyepiece 43.

In the system of the present embodiment, the broadband light source 31 has a center wavelength of 850 nm and a bandwidth of 120 nm. The high-speed linear array camera 48 acquires the OCT signal of the sample in real time and transmits the above to the signal processing module 49. Parallel light emitted from the sample arm collimating lens 38 is incident on the sample arm OCT scanning device 39. After passing through the sample arm dichroic mirror 40, the parallel light emitted from the sample arm OCT scanning device 39 is incident on the sample arm double cemented lens 42. The sample arm OCT scanning device 39 consists of two planar scanning galvanometers. The range of OCT signal acquisition is controlled by adjusting the relative positions of the two planar scanning galvanometers. The probe light emitted by the broadband light source 31 used in the device of the invention is transmitted through a fiber optic to the 80:20 fiber optic coupler 32 and split into two beams that enter the reference arm and the sample arm respectively. In particular, 80% of the probe light enters the reference arm, passes through the polarization controller 33, and is transmitted to the reference arm collimating lens 34. After collimation, the probe light passes through the dispersion matching module 35 and the reference arm focusing lens 36 and is focused on the reference arm reflecting mirror 37. Then, the beam returns to the 80:20 fiber optic coupler 32 along the original path. 20% of the probe light enters the sample arm, passes through the collimating lens 38 and the scanning galvanometer 39, and then sequentially passes through the sample arm dichroic mirror 40, the sample arm double cemented lens 42, and the eyepiece 43 before entering the sample eye 44. The beam in the sample eye 44 is focused onto the retina. The visible light stimulation source 41 emits white light with a wavelength of 360 nm to 780 nm. The stimulation light emitted thereby is coupled into the sample arm probe light path through the sample arm dichroic mirror 40, and then transmitted to the sample eye 44 after passing through the sample arm double cemented lens 42 and the eyepiece 43, thereby stimulating the optic nerve. Subsequently, the probe beam carrying the fundus information of sample eye 44 returns to the 80:20 fiber optic coupler 32 along the original path; backscattered light returned from the reference arm and sample arm interferes in the 80:20 fiber optic coupler 32. The resulting interference light passes sequentially through the collimating lens 45 and the grating 46 of the detection module, and is then focused onto the linear array camera 48 by the focusing lens 47, achieving signal detection and recording. Subsequently, the signal processing module 49 collects and further processes the signal.

The invention may obtain the microvascular hyperemia response of the retina after visible light excitation. In particular, the width, the frequency, and the amplitude of each pulse in the light stimulation pulse sequence are adjustable. In a specific embodiment, each pulse has a width of 100 ms, a frequency of 10 Hz, an amplitude of 3.0 V, and a stimulation time of 30 s, i.e., 300 pulse sequences.

FIG. 4 shows the results of the detection of retinal neurovascular coupling in healthy and substantia nigra dopaminergic neurodegenerative mice. In A of FIG. 4, the vertical axis represents the relative change in capillary blood flow volume, and the horizontal axis represents the monitoring time point, with 0 s to 30 s being the light stimulation period. It may be observed that, compared with healthy mice (n=13), the peak amplitude of retinal blood flow volume response in substantia nigra dopaminergic neurodegenerative mice (n=13) is significantly reduced, and the statistical results thereof are shown in B of FIG. 4.

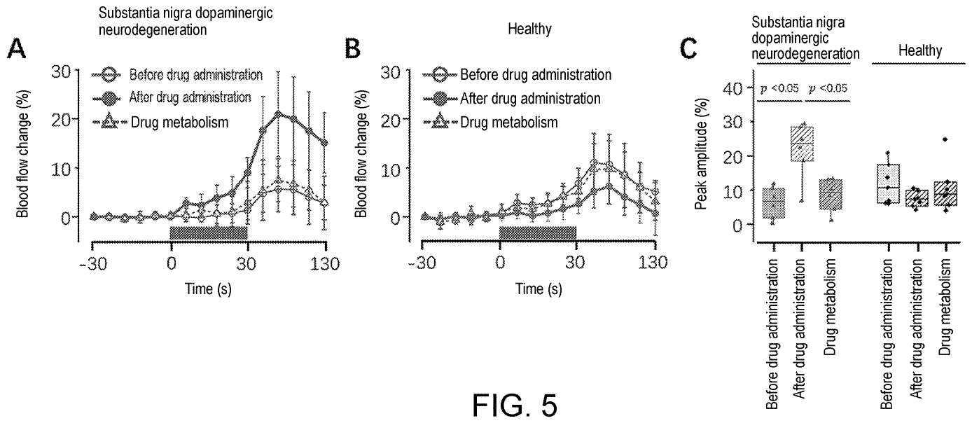

FIG. 5 shows the relative changes in retinal blood flow volume in healthy and substantia nigra dopaminergic mice before levodopa administration, after levodopa administration, and after drug metabolism.

Part A and Part B of FIG. 5 show the relative changes in retinal capillary blood flow before and after light stimulation in mice with substantia nigra dopaminergic neurodegeneration (n=6) and healthy mice (n=7), respectively, wherein the 0 s to 30 s period is the light stimulation time. It may be observed that after drug administration, the neurovascular coupling response of mice with substantia nigra dopaminergic neurodegeneration is significantly enhanced, and the neurovascular coupling response of healthy mice tended to be inhibited. This phenomenon is more intuitively reflected in the comparison of the peak amplitude of the response in Part C of FIG. 5.

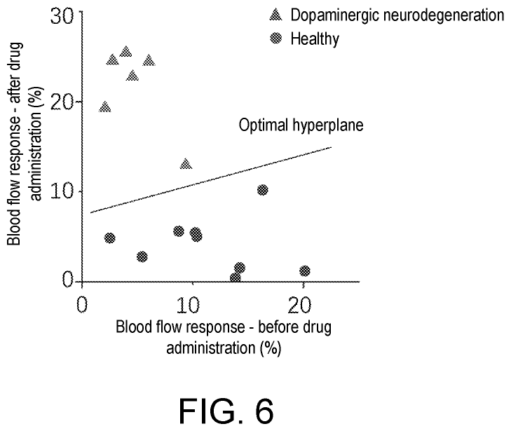

FIG. 6 shows the classification results of healthy and substantia nigra dopaminergic neurodegenerative mice based on the peak amplitude of light stimulation-induced retinal blood flow volume before and after drug administration. It may be found that the retinal neurovascular coupling function index after levodopa administration may distinguish substantia nigra dopaminergic neurodegeneration with high accuracy and high specificity.

The above experimental results fully demonstrate that the invention may combine light stimulation with the regulation of dopamine concentration in vivo, and based on the retinal neurovascular coupling mechanism, perform highly sensitive, highly specific, and highly accurate non-invasive detection of retinal functional damage caused by substantia nigra dopaminergic neurodegeneration.

Lastly, it should be noted that the above embodiments and descriptions are only used to illustrate the technical solutions of the invention and are not intended to limit them. Those skilled in the art should understand that modifications or equivalent substitutions may be made to the technical solutions of the invention without departing from the spirit and scope of the technical solutions disclosed in the invention, and such modifications or substitutions should be covered within the scope of the claims of the invention.

What is claimed is:

1. A transocular diagnostic device for Parkinson's disease caused by substantia nigra dopaminergic neurodegeneration, comprising:
an optical vision device configured to provide an optical visual stimulation to an eye of a subject before and after a specific condition of increased dopamine levels in a body of the subject, and at the same time record a stimulation index S during the optical visual stimulation and send the stimulation index S to a signal processing device;
a fundus blood flow detection device configured to detect and record fundus blood vessel and blood flow parameter change information before and after the specific condition of increased dopamine levels in the body of the subject, and before and after a visual stimulation, to generate a blood flow index B and send the blood flow index B to the signal processing device;
the signal processing device, receiving the blood flow index B from the fundus blood flow detection device and the stimulation index S from the optical vision device for analyzing and processing to obtain a retinal neurovascular coupling function index R containing dopamine-related information;
a computer or a chip receiving the retinal neurovascular coupling function index R from the signal processing device, and analyzing the retinal neurovascular coupling function index R to determine a risk or an outcome of substantia nigra dopaminergic neurodegeneration of a brain.

2. The transocular diagnostic device for Parkinson's disease caused by substantia nigra dopaminergic neurodegeneration of claim 1, wherein the optical vision device emits light and applies the light to eye of the subject through a visually perceptible means comprising a display or a lamp, thereby stimulating a retinal neural activity of the subject.

3. The transocular diagnostic device for Parkinson's disease caused by substantia nigra dopaminergic neurodegeneration of claim 1, wherein the fundus blood flow detection device records and collects the fundus blood vessel and blood flow parameter change information at least at one moment from each of a pre-stimulation stage L0 to a post-stimulation stage L1 under two conditions, before and after the specific condition of increased dopamine levels in the body of the subject, thereby generating the blood flow index B.

4. The transocular diagnostic device for Parkinson's disease caused by substantia nigra dopaminergic neurodegeneration claim 1, wherein the computer or the chip determines a risk level of dopaminergic neurodegeneration of the substantia nigra of the brain according to amplitude attenuation features or time delay features extracted from the retinal neurovascular coupling function index;
the computer or the chip determines an accurate result of substantia nigra dopaminergic neurodegeneration of the brain according to whether the amplitude attenuation features or the time delay features extracted from the retinal neurovascular coupling function index after increased dopamine levels in the body are reversible.

5. The transocular diagnostic device for Parkinson's disease caused by substantia nigra dopaminergic neurodegeneration of claim 1, wherein fundus vascular and blood flow parameters comprise a vascular size, a blood flow velocity, and a blood flow volume;
further comprising a vessel diameter and a vessel density in an OCTA image;
further comprising a mean of blood flow region decorrelation coefficients and a sum of the blood flow region decorrelation coefficients in an OCTA signal.

6. The transocular diagnostic device for Parkinson's disease caused by substantia nigra dopaminergic neurodegeneration of claim 5, wherein the stimulation index S refers to a dose of the optical visual stimulation;
the blood flow index B comprises absolute changes in fundus vessels and blood flow parameters before and after the optical visual stimulation, and/or relative changes in the fundus vessels and blood flow parameters before and after the optical visual stimulation, and/or comprises a peak amplitude, a peak time, a combination of the peak amplitude and the peak time of changes in the fundus vascular and blood flow parameters before and after the optical visual stimulation.

7. The transocular diagnostic device for Parkinson's disease caused by substantia nigra dopaminergic neurodegeneration of claim 1, wherein the retinal neurovascular coupling function index R is a combination of the blood flow index B and the stimulation index S, expressed as: R=B/S.

8. The transocular diagnostic device for Parkinson's disease caused by substantia nigra dopaminergic neurodegeneration of claim 1, wherein to determine the risk level of substantia nigra dopaminergic neurodegeneration of the brain, the control system operates in the following manner:
in a natural state DO before increased dopamine levels in the body of the subject, the optical visual stimulation is applied to the subject using the optical vision device, a retinal neurovascular coupling function index R0 under the natural state DO is calculated according to the blood flow index B obtained by the fundus blood flow detection device respectively before and after the optical visual stimulation, and the stimulation index S of the optical stimulation device, and results are then determined by the computer or the chip;
if a peak amplitude A0 in the retinal neurovascular coupling function index R0 is less than or equal to a preset first peak amplitude threshold TA1, it is determined as high risk of substantia nigra dopaminergic neurodegeneration; otherwise, it is determined as low risk;
alternatively, if a peak time TO in the retinal neurovascular coupling function index R0 is greater than or equal to a preset first peak time threshold TT1, it is determined as high risk of substantia nigra dopaminergic neurodegeneration; otherwise, it is determined as low risk;
alternatively, if the peak amplitude A0 in the retinal neurovascular coupling function index R0 is less than or equal to the preset first peak amplitude threshold TA1, and the peak time TO is greater than or equal to the preset first peak time threshold TT1, then it is determined as high risk of substantia nigra dopaminergic neurodegeneration; otherwise, it is determined as low risk.

9. The transocular diagnostic device for Parkinson's disease caused by substantia nigra dopaminergic neurodegeneration of claim 8, wherein to determine whether substantia nigra dopaminergic neurodegeneration exists in the brain, the control system continues to operate in a following manner when it is determined as high risk of substantia nigra dopaminergic neurodegeneration:
further induced under the specific condition of increased dopamine levels in the body of the subject, and then in a state D1 of increased dopamine of the subject, the subject is subjected to another optical visual stimulation by the optical vision device, then the retinal neurovascular coupling function index R1 in the state D1 of increased dopamine is calculated according to the blood flow index B obtained by the fundus blood flow detection device respectively before and after the optical visual stimulation, and the stimulation index S of the optical stimulation device by the signal processing device, and then the determination is made by the computer or the chip;
if a peak amplitude A1 in the retinal neurovascular coupling function index R1 under the state D1 of increased dopamine is greater than or equal to the preset second peak amplitude threshold TA2, then it is determined that there is substantia nigra dopaminergic neurodegeneration; otherwise, it is determined to be absent;
alternatively, if a peak time T1 in the retinal neurovascular coupling function index R1 under the state D1 of increased dopamine is less than or equal to the preset second peak time threshold TT2, then it is determined that there is substantia nigra dopaminergic neurodegeneration; otherwise, it is determined to be absent;
alternatively, if the peak amplitude A1 in the retinal neurovascular coupling function index R1 under the state D1 of increased dopamine is greater than or equal to the second peak amplitude threshold TA2, and the peak time T1 thereof is less than or equal to the second peak time threshold TT2, then it is determined that there is substantia nigra dopaminergic neurodegeneration; otherwise, it is determined to be absent.

10. A transocular diagnostic method for Parkinson's disease caused by substantia nigra dopaminergic neurodegeneration, applied to the system of claim 1, comprising:
an optical visual stimulation method of performing an optical visual stimulation on the eye of a subject before and after a specific condition of increased dopamine levels in a body of the subject, and at the same time recording a stimulation index S during the optical visual stimulation;
a fundus blood flow detection method of generating a blood flow index B by detecting fundus blood vessel and blood flow parameter change information before and after the specific condition of increased dopamine levels in the body of the subject, and before and after a visual stimulation;
a quantitative method for a retinal hyperemic response function index R of obtaining the retinal hyperemic response function index R containing dopamine-related information in combination of the blood flow index B and the stimulation index S, comprising an amplitude and a delay time;
a method for assessing a degree of substantia nigra dopaminergic neurodegeneration of assessing the degree of substantia nigra dopaminergic neurodegeneration according to the dopamine-related information contained in the retinal hyperemic response function index R.

11. The transocular diagnostic method for Parkinson's disease caused by substantia nigra dopaminergic neurodegeneration of claim 10, wherein the optical visual stimulation method is performed through a visually perceptible means comprising a display or a lamp to stimulate a retinal neural activity of the subject.

12. The transocular diagnostic method for Parkinson's disease caused by substantia nigra dopaminergic neurodegeneration of claim 10, wherein fundus vascular and blood flow parameters comprise a vascular size, a blood flow velocity, and a blood flow volume;
further comprising a vessel diameter and a vessel density in an OCTA image;
further comprising a mean of blood flow region decorrelation coefficients and a sum of the blood flow region decorrelation coefficients in an OCTA signal.

13. The transocular diagnostic method for Parkinson's disease caused by substantia nigra dopaminergic neurodegeneration of claim 10, wherein the stimulation index S specifically refers to a dose of the optical visual stimulation;
the blood flow index B comprises absolute changes in fundus vessels and blood flow parameters before and after the optical visual stimulation, and/or relative changes in the fundus vessels and blood flow parameters before and after the optical visual stimulation, and/or comprises a peak amplitude, a peak time, a combination of the peak amplitude and the peak time of changes in the fundus vascular and blood flow parameters before and after the optical visual stimulation.

14. The transocular diagnostic method for Parkinson's disease caused by substantia nigra dopaminergic neurodegeneration of claim 10, wherein the retinal neurovascular coupling function index R is a combination of the blood flow index B and the stimulation index S, expressed as: R=B/S.

15. The transocular diagnostic method for Parkinson's disease caused by substantia nigra dopaminergic neurodegeneration of claim 10, wherein the method for assessing a degree of substantia nigra dopaminergic neurodegeneration specifically comprises:
if a peak amplitude A0 in the retinal neurovascular coupling function index R0 before increased dopamine levels in the body of the subject is less than or equal to a preset first peak amplitude threshold TA1, or a peak time TO in the retinal neurovascular coupling function index R0 is greater than or equal to a preset first peak time threshold TT1, or the peak amplitude A0 in the retinal neurovascular coupling function index R0 is less than or equal to the preset first peak amplitude threshold TA1 and the peak time TO is greater than or equal to the preset first peak time threshold TT1, then it is determined as high risk of substantia nigra dopaminergic neurodegeneration, otherwise, it is determined as low risk; when it is determined as high risk of substantia nigra dopaminergic neurodegeneration, the retinal neurovascular coupling function index R1 under a state D1 of increased dopamine of the subject is further determined;
if a peak amplitude A1 in the retinal neurovascular coupling function index R1 in the state D1 of increased dopamine of the subject is greater than or equal to a preset second peak amplitude threshold TA2, or a peak time T1 in the retinal neurovascular coupling function index R1 in the state D1 of increased dopamine is less than or equal to a preset second peak time threshold TT2, or if the peak amplitude A1 in the retinal neurovascular coupling function index R1 in the state D1 of increased dopamine is greater than or equal to the second peak amplitude threshold TA2 and the peak time T1 thereof is less than or equal to the second peak time threshold TT2, then it is determined that there is substantia nigra dopaminergic neurodegeneration; otherwise, it is determined to be absent.
